SEQUENCE LISTING (5 sequences)

SEQ ID NO: 1 - DNA, 3255 bp, Artificial Sequence
  Description of Artificial Sequence Synthetic polynucleotide
accaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaaaatctacaattttatacataaccacgaattcggcgcgccctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatttaattaaggatccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatca

SEQ ID NO: 2 - DNA, 27 bp, Artificial Sequence
  Description of Artificial Sequence Synthetic primer
gtatgattgttagcatttcaaatccag

SEQ ID NO: 3 - DNA, 25 bp, Artificial Sequence
  Description of Artificial Sequence Synthetic primer
ttgagccttttccagttaggatatg

SEQ ID NO: 4 - DNA, 23 bp, Artificial Sequence
  Description of Artificial Sequence Synthetic primer
gtttatcaattatcggggcttcc

SEQ ID NO: 5 - DNA, 29 bp, Artificial Sequence
  Description of Artificial Sequence Synthetic primer
ccataacaagtagtacccttcactgataa

(19) United States
(12) Patent Application Publication
(10) Pub. No.: US 2011/0230682 A1
(43) Pub. Date: Sep. 22, 2011

(54) MICROORGANISMS WITH INACTIVATED LACTATE DEHYDROGENASE GENE (LDH) FOR CHEMICAL PRODUCTION

(75) Inventors: Matthias Schmalisch, Marlborough, MA (US); Muhammad Abdul Ali, Marlborough, MA (US); Kexue Huang, Westborough, MA (US); Tim Bowman, Amherst, MA (US)

(73) Assignee: Qteros, Inc., Marlborough, MA (US)

(21) Appl. No.: 12/729,037

(22) Filed: Mar. 22, 2010

(30) Foreign Application Priority Data
Mar. 19, 2010 (GB) GB1004631.6

(51) Int. Cl. C07C 31/08; C12N 1/21; C12P 7/06; C12P 7/16; C12P 7/04; C12P 5/02; C12P 3/00; C12N 15/63; C07H 21/04

(52) U.S. Cl. 568/840; 435/252.3; 435/161; 435/160; 435/157; 435/167; 435/168; 435/320.1; 536/24.33

(57) ABSTRACT

This invention provides systems and methods for the increased production of ethanol and other chemical compounds by recombinant Clostridium species whereby the recombinant species are genetically-engineered to disrupt lactate dehydrogenase activity and to hydrolyze and ferment carbonaceous biomass and synthesize compounds of commercial value without production of lactic acid.

### CROSS-REFERENCE

This application claims priority under 35 U.S.C. §119 to United Kingdom patent application GB1004631.6, filed Mar. 19, 2010, the disclosure of which is incorporated herein by reference in its entirety.

### BACKGROUND OF THE INVENTION

Biomass is a renewable source of energy, which can be biologically fermented to produce an end-product such as an organic acid or other useful compound. There is a growing consensus that fermenting chemicals from renewable resources such as cellulosic and lignocellulosic plant materials has great potential and can replace chemical synthesis that use petroleum reserves as energy sources, thus, reducing greenhouse gases while supporting agriculture. However, microbial fermentation requires adapting strains of microbes to industrial fermentation parameters to be economically feasible.

Clostridia species are well known as natural synthesizers of chemical products and several can adapt to commercial fermentation systems. However, few Clostridia species can saccharify and ferment biomass to commercially desirable biofuels and other chemical end products, and most of these end products are produced in low amounts. Although it is ecologically desirable to develop renewable organic substances, it is not yet economically feasible. There remains a strong need for microbial species that can consolidate the process of saccharification and fermentation in an efficient and cost-effective manner.

Under anaerobic conditions, ethanolic Clostridia sp. carry out alcoholic fermentation by the decarboxylation of pyruvate into acetaldehyde, catalysed by pyruvate dehydrogenase (PDH) and the subsequent reduction of acetaldehyde into ethanol by NADH, catalysed by alcohol dehydrogenase (ADH). In some organisms, pyruvate is also converted to lactic acid through catalysis by lactate dehydrogenase (LDH). Inactivation of LDH can result in improved ethanol yields in these organisms by directing the conversion of pyruvate to ethanol rather than lactic acid.

### SUMMARY OF THE INVENTION

In one aspect, an isolated microorganism genetically modified to lack expression of the microorganism's wild-type lactate dehydrogenase gene is provided, wherein the microorganism can hydrolyze both C5 and C6 polysaccharides. In one embodiment, said microorganism further does not express pyruvate decarboxylase. In another embodiment, said microorganism is a Clostridium species. In another embodiment, said Clostridium species is C. phytofermentans. In another embodiment, said microorganism is a mesophilic microorganism. In another embodiment, said microorganism does not comprise an integration element in the lactate dehydrogenase gene.

In another aspect, an isolated microorganism is provided selected from the group consisting of: NRRL Accession No. NRRL B-B-50351, NRRL B-50352, NRRL B-50353, NRRL B-503554, NRRL B-50355, NRRL B-50356, NRRL B-50357, NRRL B-50358, and NRRL B-50359.

In another aspect, a mesophilic microorganism that lacks lactate dehydrogenase activity is provided, wherein said mesophilic microorganism ferments polysaccharides.

In another aspect, a mesophilic microorganism is provided, genetically modified to produce higher amounts of ethanol, wherein said modification comprises inactivation of lactic acid synthesis as it occurs in the wild-type mesophilic microorganism.

In another aspect, a product for production of a chemical product is provided comprising: a fermentation vessel comprising a carbonaceous biomass; and a microorganism genetically modified to inactivate a lactate dehydrogenase gene of the wild-type organism, wherein the microorganism hydrolyzes both C5 and C6 polysaccharides and produces a chemical product. In one embodiment, said microorganism is capable of direct fermentation of C5 and C6 carbohydrates. In another embodiment, said microorganism is a mesophilic microorganism. In another embodiment, said microorganism is a Clostridium species. In another embodiment, said lactate dehydrogenase gene is Cphy_{—}1232 or Cphy_{—}1117. In another embodiment, said biomass comprises cellulosic or lignocellulosic materials. In another embodiment, said biomass comprises one or more of corn steep solids, corn steep liquor, malt syrup, xylan, cellulose, hemicellulose, fructose, glucose, mannose, rhamnose, or xylose. In another embodiment, said biomass comprises municipal waste, wood, plant material, plant material extract, bagasse, switch grass, algae, corn stover, poplar, a natural or synthetic polymer, or a combination thereof. In another embodiment, said biomass is pre-conditioned with acid treatment. In another embodiment, said chemical product is an alcohol, a gas, an acid, a fatty acid, an isoprenoid, or a polyisoprene. In another embodiment, said microorganism is capable of hydrolyzing xylose. In another embodiment, said chemical product is ethanol, butanol, methanol, methane, hydrogen or rubber.

In another aspect, a process for producing a chemical product is provided comprising: contacting a carbonaceous biomass with a genetically modified microorganism that is capable of direct hydrolysis and fermentation of said biomass, wherein said microorganism has a reduced capacity to synthesize lactic acid compared to the wild-type microorganism; and allowing sufficient time for said hydrolysis and fermentation to produce the chemical product. In one embodiment, said microorganism is modified to enhance production of a fermentive end-product is selected from the group consisting of Clostridium phytofermentans, Thermoanaerobacter ethanolicus, Clostridium thermocellum, Clostridium beijerinckii, Clostridium acetobutylicum, Clostridium. cellovorans, Clostridium tyrobutyricum, Clostridium thermobutyricum, Thermoanaerobacterium saccharolyticum, Thermoanaerobacter thermohydrosulfuricus, Saccharomyces cerevisiae, Clostridium acetobutylicum, Moorella ssp., Carboxydocella ssp., Zymomonas mobilis, E. coli, and Klebsiella oxytoca. In another embodiment, said microorganism is capable of direct fermentation of C5 and C6 carbohydrates. In another embodiment, said microorganism is a Clostridium. In another embodiment, said microorganism is a mesophilic microorganism. In another embodiment, said microorganism is C. phytofermentans. In another embodiment, said biomass comprises cellulosic or lignocellulosic materials. In another embodiment, said biomass comprises one or more of corn steep solids, corn steep liquor, malt syrup, xylan, cellulose, hemicellulose, fructose, glucose, mannose, rhamnose, or xylose. In another embodiment, said biomass comprises municipal waste, wood, plant material, plant material extract, bagasse, switch grass, algae, corn stover, poplar, a natural or synthetic polymer, or a combination thereof. In another embodiment, said biomass is pre-conditioned with acid treatment. In another embodiment, said process occurs at a temperature between 10° C. and 35° C.

In another embodiment, a chemical product produced by the process of claim 22 is provided. In another embodiment, said product is an alcohol. In another embodiment, said alcohol is ethanol.

In another aspect, an isolated micoorganism that ferments cellulosic or lignocellulosic materials to produce ethanol in a concentration that is at least 90% of a theoretical yield is provided, wherein the micoorganism is a recombinant organism that produces less lactic acid than the wild-type micoorganism. In another embodiment, the cellulolytic substrate is a lignocellulosic material. In another embodiment, the micoorganism is Gram negative.

In another aspect, a plasmid comprising SEQ ID NO:1 is provided.

In another aspect, a primer selected from the group comprising SEQ ID NO:2, SEQ ID NO:3, SEQ ID NO:4, and SEQ ID NO:5 is provided.

In another aspect, a plasmid comprising a nucleic acid sequence with 70-99.9% similarity to SEQ ID NO:1 is provided. In one embodiment a plasmid comprising a nucleic acid sequence with 70% similarity to SEQ ID NO:1 is provided. In one embodiment a plasmid comprising a nucleic acid sequence with 75% similarity to SEQ ID NO:1 is provided. In one embodiment a plasmid comprising a nucleic acid sequence with 80% similarity to SEQ ID NO:1 is provided. In one embodiment a plasmid comprising a nucleic acid sequence with 85% similarity to SEQ ID NO:1 is provided. In one embodiment a plasmid comprising a nucleic acid sequence with 90% similarity to SEQ ID NO:1 is provided. In one embodiment a plasmid comprising a nucleic acid sequence with 95% similarity to SEQ ID NO:1 is provided. In one embodiment a plasmid comprising a nucleic acid sequence with 99% similarity to SEQ ID NO:1 is provided. In one embodiment a plasmid comprising a nucleic acid sequence with 99.9% similarity to SEQ ID NO:1 is provided.

In another aspect, a primer comprising a nucleic acid sequence with 70-99.9% similarity to SEQ ID NO:2, SEQ ID NO:3, SEQ ID NO:4, or SEQ ID NO:5 is provided. In one embodiment a primer comprising a nucleic acid sequence with 70% similarity to SEQ ID NO:2, SEQ ID NO:3, SEQ ID NO:4, or SEQ ID NO:5 is provided. In one embodiment a primer comprising a nucleic acid sequence with 75% similarity to SEQ ID NO:2, SEQ ID NO:3, SEQ ID NO:4, or SEQ ID NO:5 is provided. In one embodiment a primer comprising a nucleic acid sequence with 80% similarity to SEQ ID NO:2, SEQ ID NO:3, SEQ ID NO:4, or SEQ ID NO:5 is provided. In one embodiment a primer comprising a nucleic acid sequence with 85% similarity to SEQ ID NO:2, SEQ ID NO:3, SEQ ID NO:4, or SEQ ID NO:5 is provided. In one embodiment a primer comprising a nucleic acid sequence with 90% similarity to SEQ ID NO:2, SEQ ID NO:3, SEQ ID NO:4, or SEQ ID NO:5 is provided. In one embodiment a primer comprising a nucleic acid sequence with 95% similarity to SEQ ID NO:2, SEQ ID NO:3, SEQ ID NO:4, or SEQ ID NO:5 is provided. In one embodiment a primer comprising a nucleic acid sequence with 99% similarity to SEQ ID NO:2, SEQ ID NO:3, SEQ ID NO:4, or SEQ ID NO:5 is provided. In one embodiment a primer comprising a nucleic acid sequence with 99.9% similarity to SEQ ID NO:2, SEQ ID NO:3, SEQ ID NO:4, or SEQ ID NO:5 is provided.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a representation of several end-products synthesized from pyruvate.

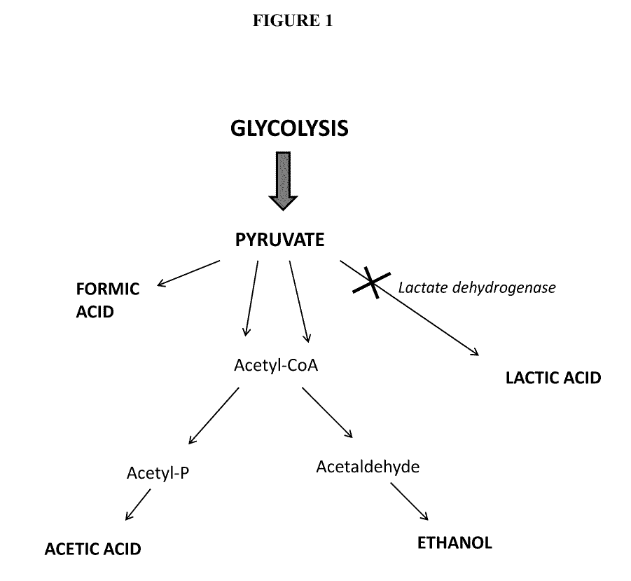

FIG. 2 depicts the primers designed for inactivating LDH genes.

FIG. 3 depicts plasmids containing Cphy_{—}1232 and Cphy_{—}1117 cloned fragments.

FIG. 4 depicts the pQSeq plasmid.

FIG. 5 depicts the pQSeq plasmid comprising Cphy_{—}1232 and Cphy_{—}1117 cloned fragments.

FIG. 6 is an example of a method for producing a chemical product from biomass by first treating biomass with an acid at elevated temperature and pressure in a hydrolysis unit.

FIG. 7 depicts a method for producing a chemical product from biomass by charging biomass to a fermentation vessel.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### DETAILED DESCRIPTION OF THE INVENTION

The following description and examples illustrate embodiments of the present invention in detail. It is to be understood that this invention is not limited to the particular methodology, protocols, cell lines, constructs and reagents described herein and as such can vary. Those of skill in the art will recognize that there are numerous variations and modifications of this invention that are encompassed within its scope.

The invention comprises methods and compositions directed to saccharification and fermentation of various biomass substrates to desired products.

In one embodiment, products include modified strains of microorganisms, including algae, fungi, gram-positive and gram-negative bacteria, including species of Clostridium, including C. phytofermentans that can be used in production of chemicals from lignocellulosic, cellulosic, hemicellulosic, algal and other plant-based feedstocks or plant polysaccharides. Products further include the chemical compounds, fermentive-end products, biofuels and the like from the processes using these modified organisms. Described herein are also methods of producing chemical compounds, fermentive-end products, biofuels and the like using these referenced microorganisms.

In another embodiment, organisms are genetically-modified strains of bacteria, including Clostridium sp., including C. phytofermentans. Bacteria comprising altered expression or structure of a gene or genes relative to the original organisms strain, wherein such genetic modifications result in increased efficiency of chemical production. In some embodiments, the genetic modifications are introduced by genetic recombination. In some embodiments, the genetic modifications are introduced by nucleic acid transformation. In further embodiments, the genetic modifications encompass inactivation of one or more genes of Clostridium sp., including C. phytofermentans through any number of genetic methods, including but not limited to single-crossover or double-crossover gene replacement, transposable element insertion, integrational plasmid technology (e.g., using non-replicative or replicative integrative plasmids), targeted gene inactivation using group II intron-based Targetron technology (Chen Y. et al. (2005) Appl Environ Microbial 71:7542-7547), or targeted gene inactivation using ClosTron Group II intron directed mutagenesis (Heap J T et al. (2010) J. Microbiol Methods 80:49-55. The restriction and modification system of a Clostridium sp. can be modified to increase the efficiency of transformation with unmethylated DNA (Dong H. et al. (2010) PLOS One 5(2): e9038). Interspecific conjugation (for example, with E. coli), can be used to transfer nucleic acid into a Clostridium sp. (Tolonen A C et al. (2009) Molecular Microbiology, 74: 1300-1313). In some strains, genetic modification can comprise inactivation of one or more endogenous nucleic acid sequence(s) and also comprise introduction and activation of heterologous or exogenous nucleic acid sequence(s) and promoters.

In some variations, the recombinant C. phytofermentans organisms described herein comprise a heterologous nucleic acid sequence. In some variations, the recombinant C. phytofermentans comprise one or more introduced heterologous nucleic acid(s). In some embodiments, the heterologous nucleic acid sequence is controlled by an inducible promoter. In some variations, expression of the heterologous nucleic acid sequence is controlled by a constitutive promoter.

The discovery that C. phytofermentans microbes can produce a variety of chemical products is a great advantage over other fermenting organisms. C. phytofermentans is capable of simultaneous hydrolysis and fermentation of a variety of feedstocks comprised of cellulosic, hemicellulosic or lignocellulosic materials, thus eliminating or drastically reducing the need for hydrolysis of polysaccharides prior to fermentation of sugars. Further, C. phytofermentans utilizes both hexose and pentose polysaccharides and sugars, producing a highly efficient yield from feedstocks.

Another advantage of C. phytofermentans is its ability to ferment oligomers, resulting in a great cost savings for processors that have to pretreat biomass prior to fermentation. To produce a stream of monosaccharides for most fermenting organisms such as yeasts, that cannot ferment oligomers or polymeric saccharides, harsh prolonged pretreatment is required. This results in higher costs due to the chemical and energy requirements and to the loss of sugars during the pretreatment, as well as the increased production of breakdown products and inhibitors. Because C. phytofermentans can hydrolyze polysaccharides and ferment oligomers, it does not require severe biomass pretreatment resulting in a higher conversion efficiency of carbohydrate in biomass and increased yields at reduced costs.

### DEFINITIONS

Unless characterized differently, technical and scientific terms used herein have the same meaning as commonly understood by one of ordinary skill in the art to which this invention belongs.

The term “about” as used herein, refers to a range that is 15% plus or minus from a stated numerical value within the context of the particular usage. For example, about 10 would include a range from 8.5 to 11.5.

By “enzyme reactive conditions” it is meant that any necessary conditions are available in an environment (i.e., such factors as temperature, pH, lack of inhibiting substances) which will permit the enzyme to function. Enzyme reactive conditions can be either in vitro, such as in a test tube, or in vivo, such as within a cell.

As used herein, the terms “function” and “functional” and the like refer to a biological or enzymatic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences). The term “host cell” includes an individual cell or cell culture which can be or has been a recipient of any recombinant vector(s) or isolated polynucleotide of the invention. Host cells include progeny of a single host cell, and the progeny can not necessarily be completely identical (in morphology or in total DNA complement) to the original parent cell due to natural, accidental, or deliberate mutation and/or change. A host cell includes cells transfected, transformed, or infected in vivo or in vitro with a recombinant vector or a polynucleotide of the invention. A host cell which comprises a recombinant vector of the invention is a recombinant host cell, recombinant cell, or recombinant microorganism.

By “isolated” is meant material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

By “increased” or “increasing” is meant the ability of one or more recombinant microorganisms to produce a greater amount of a given product or molecule (e.g., commodity chemical, biofuel, or intermediate product thereof) as compared to a control microorganism, such as an unmodified microorganism or a differently modified microorganism. An “increased” amount is typically a “statistically significant” amount, and can include an increase that is 1, 2, 3, 4, 5, 6, 7, 8, 9, 10, 15, 20, 30 or more times (including all integers and decimal points in between, e.g., 1.5, 1.6, 1.7. 1.8, etc.) the amount produced by an unmodified microorganism or a differently modified microorganism.

The term “operably linked” as used herein means placing a gene under the regulatory control of a promoter, which then controls the transcription and optionally the translation of the gene. In one example for the construction of promoter/structural gene combinations, the genetic sequence or promoter is positioned at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, a regulatory sequence element can be positioned with respect to a gene to be placed under its control in the same position as the element is situated in its in its natural setting with respect to the native gene it controls.

The term “constitutive promoter” refers to a polynucleotide sequence that induces transcription or is typically active, (i.e., promotes transcription), under most conditions, such as those that occur in a host cell. A constitutive promoter is generally active in a host cell through a variety of different environmental conditions.

The term “inducible promoter” refers to a polynucleotide sequence that induces transcription or is typically active only under certain conditions, such as in the presence of a specific transcription factor or transcription factor complex, a given molecule factor (e.g., IPTG) or a given environmental condition (e.g., CO₂ concentration, nutrient levels, light, heat). In the absence of that condition, inducible promoters typically do not allow significant or measurable levels of transcriptional activity.

The terms “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, a polynucleotide sequences can include genomic sequences, extra-genomic and plasmid-encoded sequences and smaller engineered gene segments that express, or can be adapted to express, proteins, polypeptides, peptides and the like. Such segments can be naturally isolated, or modified synthetically by the hand of man.

Polynucleotides can be single-stranded (coding or antisense) or double-stranded, and can be DNA (genomic, cDNA or synthetic) or RNA molecules. Additional coding or non-coding sequences can, but need not, be present within a polynucleotide of the present invention, and a polynucleotide can, but need not, be linked to other molecules and/or support materials.

Polynucleotides can comprise a native sequence (i.e., an endogenous sequence) or can comprise a variant, or a biological functional equivalent of such a sequence. Polynucleotide variants can contain one or more substitutions, additions, deletions and/or insertions, as further described below. In one embodiment a polynucleotide variant encodes a polypeptide with the same sequence as the native protein. In another embodiment a polynucleotide variant encodes a polypeptide with substantially similar enzymatic activity as the native protein. In another embodiment a polynucleotide variant encodes a protein with increased enzymatic activity relative to the native polypeptide. The effect on the enzymatic activity of the encoded polypeptide can generally be assessed as described herein.

A polynucleotide, regardless of the length of the coding sequence itself, can be combined with other DNA sequences, such as promoters, polyadenylation signals, additional restriction enzyme sites, multiple cloning sites, other coding segments, and the like, such that their overall length can vary considerably. It is therefore contemplated that a polynucleotide fragment of almost any length can be employed, with the total length preferably being limited by the ease of preparation and use in the intended recombinant DNA protocol.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein.

The terms “polynucleotide variant” and “variant” also include naturally-occurring allelic variants that encode these enzymes. Examples of naturally-occurring variants include allelic variants (same locus), homologs (different locus), and orthologs (different organism). Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having a suitable enzymatic activity described herein (e.g., C—C ligase, diol dehyodrogenase, pectate lyase, alginate lyase, diol dehydratase, transporter, etc.).

Non-naturally occurring variants can be made by mutagenesis techniques, including those applied to polynucleotides, cells, or microorganisms. The variants can contain nucleotide substitutions, deletions, inversions and insertions. Variation can occur in either or both the coding and non-coding regions. In certain aspects, non-naturally occurring variants can have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For nucleotide sequences, conservative variants include those sequences that, because of the degeneracy of the genetic code, encode the amino acid sequence of a reference polypeptide. Variant nucleotide sequences also include synthetically derived nucleotide sequences, such as those generated, for example, by using site-directed mutagenesis but which still encode a biologically active polypeptide. Generally, variants of a particular reference nucleotide sequence will have at least about 30%, 40% 50%, 55%, 60%, 65%, 70%, generally at least about 75%, 80%, 85%, 90% to 95% or more, and even about 97% or 98% or more sequence identity to that particular nucleotide sequence as determined by sequence alignment programs described elsewhere herein using default parameters.

As used herein, the term “hybridizes under low stringency, medium stringency, high stringency, or very high stringency conditions” describes conditions for hybridization and washing. Guidance for performing hybridization reactions can be found in Ausubel et al., “Current Protocols in Molecular Biology”, John Wiley & Sons Inc, 1994-1998, Sections 6.3.1-6.3.6. Aqueous and non-aqueous methods are described in that reference and either can be used.

Reference herein to “low stringency” conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also can include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency” conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also can include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

“High stringency” conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also can include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

Due to the degeneracy of the genetic code, amino acids can be substituted for other amino acids in a protein sequence without appreciable loss of the desired activity (see Table 1 below). It is thus contemplated that various changes can be made in the peptide sequences of the disclosed protein sequences, or their corresponding nucleic acid sequences without appreciable loss of the biological activity.

In making such changes, the hydropathic index of amino acids can be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Amino acids have been assigned a hydropathic index on the basis of their hydrophobicity and charge characteristics. These are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids can be substituted by other amino acids having a similar hydropathic index or score and result in a protein with similar biological activity, i.e., still obtain a biologically-functional protein. In one embodiment, the substitution of amino acids whose hydropathic indices are within +/−0.2 is preferred, those within +/−0.1 are more preferred, and those within +/−.0.5 are most preferred.

It is also understood in the art that the substitution of like amino acids can be made effectively on the basis of hydrophilicity. U.S. Pat. No. 4,554,101 (Hopp, which is herein incorporated by reference in its entirety) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0.+−0.1); serine (+0.3); asparagine/glutamine (+0.2); glycine (0); threonine (−0.4); proline (−0.5.+−0.1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid can be substituted by another amino acid having a similar hydrophilicity score and still result in a protein with similar biological activity, i.e., still obtain a biologically functional protein. In one embodiment the substitution of amino acids whose hydropathic indices are within +/−0.2 is preferred, those within +/−0.1 are more preferred, and those within .+/−.0.5 are most preferred.

As outlined above, amino acid substitutions can be based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous can also be used if these resulting proteins have the same or improved characteristics, relative to the unmodified polypeptide from which they are engineered.

In one embodiment a polynucleotide comprises codons in its protein coding sequence that are optimized to increase the thermostability of an mRNA transcribed from the polynucleotide. In one embodiment this optimization does not change the amino acid sequence encoded by the polynucleotide. In another embodiment a polynucleotide comprises codons in its protein coding sequence that are optimized to increase translation efficiency of an mRNA from the polynucleotide in a host cell. In one embodiment this optimization does not change the amino acid sequence encoded by the polynucleotide.

The RNA codon table below (Table 1) shows the 64 codons and the amino acid for each. The direction of the mRNA is 5′ to 3′.

TABLE 1

| | | 2nd base | | | |
|---|---|---|---|---|---|
| | | U | C | A | G |
| 1st | U | UUU (Phe/F) | UCU (Ser/S) | UAU (Tyr/Y) | UGU (Cys/C) |
| base | | Phenylalanine | Serine | Tyrosine | Cysteine |
| | | UUC (Phe/F) | UCC (Ser/S) | UAC (Tyr/Y) | UGC (Cys/C) |
| | | Phenylalanine | Serine | Tyrosine | Cysteine |
| | | UUA (Leu/L) | UCA (Ser/S) | UAA Ochre | UGA Opal |
| | | Leucine | Serine | (Stop) | (Stop) |
| | | UUG (Leu/L) | UCG (Ser/S) | UAG Amber | UGG (Trp/W) |
| | | Leucine | Serine | (Stop) | Tryptophan |
| | C | CUU (Leu/L) | CCU (Pro/P) | CAU (His/H) | CGU (Arg/R) |
| | | Leucine | Proline | Histidine | Arginine |
| | | CUC (Leu/L) | CCC (Pro/P) | CAC (His/H) | CGC (Arg/R) |
| | | Leucine | Proline | Histidine | Arginine |
| | | CUA (Leu/L) | CCA (Pro/P) | CAA (Gln/Q) | CGA (Arg/R) |
| | | Leucine | Proline | Glutamine | Arginine |
| | | CUG (Leu/L) | CCG (Pro/P) | CAG (Gln/Q) | CGG (Arg/R) |
| | | Leucine | Proline | Glutamine | Arginine |
| | A | AUU (Ile/I) | ACU (Thr/T) | AAU (Asn/N) | AGU (Ser/S) |
| | | Isoleucine | Threonine | Asparagine | Serine |
| | | AUC (Ile/I) | ACC (Thr/T) | AAC (Asn/N) | AGC (Ser/S) |
| | | Isoleucine | Threonine | Asparagine | Serine |
| | | AUA (Ile/I) | ACA (Thr/T) | AAA (Lys/K) | AGA (Arg/R) |
| | | Isoleucine | Threonine | Lysine | Arginine |
| | | AUG^{[A]} (Met/M) | ACG (Thr/T) | AAG (Lys/K) | AGG (Arg/R) |
| | | Methionine | Threonine | Lysine | Arginine |
| | G | GUU (Val/V) | GCU (Ala/A) | GAU (Asp/D) | GGU (Gly/G) |
| | | Valine | Alanine | Aspartic acid | Glycine |
| | | GUC (Val/V) | GCC (Ala/A) | GAC (Asp/D) | GGC (Gly/G) |
| | | Valine | Alanine | Aspartic acid | Glycine |
| | | GUA (Val/V) | GCA (Ala/A) | GAA (Glu/E) | GGA (Gly/G) |
| | | Valine | Alanine | Glutamic acid | Glycine |
| | | GUG (Val/V) | GCG (Ala/A) | GAG (Glu/E) | GGG (Gly/G) |
| | | Valine | Alanine | Glutamic acid | Glycine |

^{[A]}The codon AUG both codes for methionine and serves as an initiation site: the first AUG in an mRNA's coding region is where translation into protein begins.

The present invention contemplates the use in the methods described herein of variants of full-length polypeptides having any of the enzymatic activities described herein, truncated fragments of these full-length polypeptides, variants of truncated fragments, as well as their related biologically active fragments. Typically, biologically active fragments of a polypeptide can participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a polypeptide/enzyme an enzymatic activity described herein include peptides comprising amino acid sequences sufficiently similar to, or derived from, the amino acid sequences of a (putative) full-length reference polypeptide sequence. Typically, biologically active fragments comprise a domain or motif with at least one enzymatic activity, and can include one or more (and in some cases all) of the various active domains. A biologically active fragment of a an enzyme can be a polypeptide fragment which is, for example about 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, 50% of an activity of the wild-type polypeptide from which it is derived.

The term “exogenous” as used herein, refers to a polynucleotide sequence or polypeptide that does not naturally occur in a given wild-type cell or microorganism, but is typically introduced into the cell by a molecular biological technique, i.e., engineering to produce a recombinant microorganism. Examples of “exogenous” polynucleotides include vectors, plasmids, and/or man-made nucleic acid constructs encoding a desired protein or enzyme.

The term “endogenous” as used herein, refers to naturally-occurring polynucleotide sequences or polypeptides that can be found in a given wild-type cell or microorganism. For example, certain naturally-occurring bacterial or yeast species do not typically contain a benzaldehyde lyase gene, and, therefore, do not comprise an “endogenous” polynucleotide sequence that encodes a benzaldehyde lyase. In this regard, it is also noted that even though a microorganism can comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the of pathways, genes, or enzymes described herein can utilize or rely on an “endogenous” sequence, or can be provided as one or more “exogenous” polynucleotide sequences, and/or can be used according to the endogenous sequences already contained within a given microorganism.

The term “sequence identity” for example, comprising a “sequence 50% identical to,” as used herein, refers to the extent that sequences are identical on a nucleotide-by-nucleotide basis or an amino acid-by-amino acid basis over a window of comparison. Thus, a “percentage of sequence identity” can be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides can each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window can comprise additions or deletions (i.e., gaps) of about 20% or less as compared to the reference sequence (which does not comprise additions or deletions) for optimal alignment of the two sequences. Optimal alignment of sequences for aligning a comparison window can be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also can be made to the BLAST family of programs as for example disclosed by Altschul et al., 1997, Nucl. Acids Res. 25:3389, which is herein incorporated by reference in its entirety. A detailed discussion of sequence analysis can be found in Unit 19.3 of Ausubel et al., “Current Protocols in Molecular Biology”, John Wiley & Sons Inc, 1994-1998, Chapter 15, which is herein incorporated by reference in its entirety.

The term “transformation” as used herein, refers to the permanent, heritable alteration in a cell resulting from the uptake and incorporation of foreign DNA into the host-cell genome. This includes the transfer of an exogenous gene from one microorganism into the genome of another microorganism as well as the addition of additional copies of an endogenous gene into a microorganism.

The term “recombinant” means an organism is genetically modified to comprise one or more heterologous or endogenous nucleic acid molecules, such as in a plasmid or vector. Such nucleic acid molecules can be comprised extrachromosomally or integrated into the chromosome of an organism. The term “non-recombinant” means an organism is not genetically modified. For example, a recombinant organism can be modified to overexpress an endogenous gene encoding an enzyme through modification of promoter elements (e.g., replacing an endogenous promoter element with a constitutive or highly active promoter). Alternatively, a recombinant organism can be modified by introducing a heterologous nucleic acid molecule encoding a protein that is not otherwise expressed in the host organism.

The term “vector” as used herein, refers to a polynucleotide molecule, such as a DNA molecule. It can be derived, from a plasmid, bacteriophage, yeast or virus, into which a polynucleotide can be inserted or cloned. A vector can contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector can comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. A vector can be one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The terms “wild-type” and “naturally-occurring” as used herein are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene. Many gene loci have different allelic forms. The form that is not most frequently observed in a population can be referred to as a mutant form.

The term “fuel” or “biofuel” as used herein has its ordinary meaning as known to those skilled in the art and can include one or more compounds suitable as liquid fuels, gaseous fuels, biodiesel fuels (long-chain alkyl (methyl, propyl or ethyl) esters), heating oils (hydrocarbons in the 14-20 carbon range), reagents, chemical feedstocks and includes, but is not limited to, hydrocarbons (both light and heavy), hydrogen, methane, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The terms “fermentation end-product” or “end-product” as used herein has its ordinary meaning as known to those skilled in the art and can include one or more biofuels, chemical additives, processing aids, food additives, organic acids (e.g. acetic, lactic, formic, citric acid etc.), derivatives of organic acids such as esters (e.g. wax esters, glycerides, etc.) or other functional compounds. These end-products include, but are not limited to, an alcohol, ethanol, butanol, methanol, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases and can be present as a pure compound, a mixture, or an impure or diluted form.

Various end-products can be produced through saccharification and fermentation using enzyme-enhancing products and processes. Examples of end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, dodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-) butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, isoprenoids, and polyisoprenes, including rubber.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The cellular activity, including cell growth can be growing aerobic, microaerophilic, or anaerobic. The cells can be in any phase of growth, including lag (or conduction), exponential, transition, stationary, death, dormant, vegetative, sporulating, etc.

The term “external source” as it relates to a quantity of an enzyme or enzymes provided to a product or a process means that the quantity of the enzyme or enzymes is not produced by a microorganism in the product or process. An external source of an enzyme can include, but is not limited to an enzyme provided in purified form, cell extracts, culture medium or an enzyme obtained from a commercially available source.

The term “plant polysaccharide” as used herein has its ordinary meaning as known to those skilled in the art and can comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or non-regular pattern. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be used as a carbon source by the microorganism, including monomers, dimers, and polymers of these compounds including two or more of these compounds. In some cases, the microorganism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be used by the microorganism at hand. For some microorganisms, this would include conversion to monosaccharides, disaccharides, trisaccharides, and oligosaccharides of up to about seven monomer units, as well as similar sized chains of sugar derivatives and combinations of sugars and sugar derivatives. For some microorganisms, the allowable chain-length can be longer (e.g. 8, 9, 10, 11, 12, 13, 14, 15, 16, 17, 18, 19, or 20 monomer units or more) and for some microorganisms the allowable chain-length can be shorter (e.g. 1, 2, 3, 4, 5, 6, or 7 monomer units).

The term “carbonaceous biomass” as used herein has its ordinary meaning as known to those skilled in the art and can include one or more biological material that can be converted into a biofuel, chemical or other product. Carbonaceous biomass can comprise municipal waste, wood, plant matter, plant extract, a natural or synthetic polymer, or a combination thereof.

Plant matter can include, but is not limited to, woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, bamboo, algae and material derived from these.

“Biomass” can include, but is not limited to, woody or non-woody plant matter, aquatic or marine biomass, fruit-based biomass such as fruit waste, and vegetable-based biomass such as vegetable waste, and animal based biomass among others. Examples of aquatic or marine biomass include, but are not limited to, kelp, other seaweed, algae, and marine microflora, microalgae, sea grass, salt marsh grasses such as Spartina sp. or Phragmites sp. and the like. In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

Examples of fruit and/or vegetable biomass include, but are not limited to, any source of pectin such as plant peel and pomace including citrus, orange, grapefruit, potato, tomato, grape, mango, gooseberry, carrot, sugar-beet, and apple, among others. In one embodiment plant matter is characterized by the chemical species present, such as proteins, polysaccharides and oils. In one embodiment plant matter includes agricultural waste byproducts or side streams such as pomace, corn steep liquor, corn steep solids, distillers grains, peels, pits, fermentation waste, straw, lumber, sewage, garbage and food leftovers. These materials can come from farms, forestry, industrial sources, households, etc. In another embodiment biomass comprises animal matter, including, for example milk, meat, fat, animal processing waste, and animal waste. The term “feedstock” is frequently used to refer to biomass being used for a process, such as those described herein.

Examples of polysaccharides, oligosaccharides, monosaccharides or other sugar components of biomass include, but are not limited to, alginate, agar, carrageenan, fucoidan, floridean starch, pectin, gluronate, mannuronate, mannitol, lyxose, cellulose, hemicellulose, glycerol, xylitol, glucose, mannose, galactose, xylose, xylan, mannan, arabinan, arabinose, glucuronate, galacturonate (including di- and tri-galacturonates), rhamnose, and the like.

The term “broth” as used herein has its ordinary meaning as known to those skilled in the art and can include the entire contents of the combination of soluble and insoluble matter, suspended matter, cells and medium, such as for example the entire contents of a fermentation reaction can be referred to as a fermentation broth.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity (e.g. g/L/d) is different from “titer” (e.g. g/L) in that productivity includes a time term, and titer is analogous to concentration.

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as: C₆H₁₂O₆→2C₂H₅OH+2 CO₂ and the theoretical maximum conversion efficiency or yield is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification yield. In one embodiment, given carbon source comprising 10 g of cellulose, the theoretical maximum conversion efficiency can be calculated by assuming saccharification of the cellulose to the assimilable carbon source glucose of about 75% by weight. In this embodiment, 10 g of cellulose can provide 7.5 g of glucose which can provide a maximum theoretical conversion efficiency of about 7.5 g*51% or 3.8 g of ethanol. In other cases, the efficiency of the saccharification step can be calculated or determined, i.e., saccharification yield. Saccharification yields can include between about 10-100%, about 20-90%, about 30-80%, about 40-70% or about 50-60%, such as about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% for any carbohydrate carbon sources larger than a single monosaccharide subunit.

The terms “pretreatment” or “pretreated” as used herein refer to any mechanical, chemical, thermal, biochemical process or combination of these processes whether in a combined step or performed sequentially, that achieves disruption or expansion of a biomass so as to render the biomass more susceptible to attack by enzymes and/or microbes. In some embodiments, pretreatment can include removal or disruption of lignin so is to make the cellulose and hemicellulose polymers in the plant biomass more available to cellulolytic enzymes and/or microbes, for example, by treatment with acid or base. In some embodiments, pretreatment can include the use of a microorganism of one type to render plant polysaccharides more accessible to microorganisms of another type. In some embodiments, pretreatment can also include disruption or expansion of cellulosic and/or hemicellulosic material. Steam explosion, and ammonia fiber expansion (or explosion) (AFEX) are well known thermal/chemical techniques. Hydrolysis, including methods that utilize acids and/or enzymes can be used. Other thermal, chemical, biochemical, enzymatic techniques can also be used.

The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

The term “recombinant” as used herein, refers to a microorganism is genetically modified to comprise one or more heterologous or endogenous nucleic acid molecules. Such nucleic acid molecules can be comprised extrachromosomally or integrated into the chromosome of a microorganism. The term “non-recombinant” means a microorganism is not genetically modified. For example, a recombinant microorganism can be modified to overexpress an endogenous gene encoding an enzyme through modification of promoter elements (e.g., replacing an endogenous promoter element with a constitutive or highly active promoter). Alternatively, a recombinant microorganism can be modified by introducing a heterologous or another copy of an endogenous nucleic acid molecule encoding a protein that is not otherwise expressed in the host microorganism.

The term “sugar compounds” as used herein has its ordinary meaning as known to those skilled in the art and can include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

Generally, compositions and methods are provided for enzyme conditioning of feedstock or biomass to allow saccharification and fermentation to one or more industrially useful fermentive end-products.

### Modification of Microbe

Any enzyme can be selected from the annotated genome of C. phytofermentans, another bacterial species, such as B. subtilis, E. coli, various Clostridium species, or yeasts such as S. cerevisiae for utilization in products and processes described herein. Examples include enzymes such as L-butanediol dehydrogenase, acetoin reductase, 3-hydroxyacyl-CoA dehydrogenase, cis-aconitate decarboxylase or the like, to create pathways for new products from biomass.

Furthermore, a microorganism, such as C. phytofermentans can be modified to enhance production of one or more enzymes necessary to accumulate new products. This is accomplished through over expression of C. phytofermentans endogenous enzymes or through expression of heterologous or exogenous genes in C. phytofermentans. For hetereologous expression bacteria or yeast can be modified through recombinant technology. (e.g., Brat et al. Appl. Env. Microbio. 2009; 75(8):2304-2311, disclosing expression of xylose isomerase in S. cerevisiae). Product production can also be enhanced by knock-outs of C. phytofermentans endogenous genes by methods such as RNAi or antisense technology and the like known to those of skill in the art.

A variety of promoters (e.g., constitutive promoters, inducible promoters) can be utilized to drive expression of the heterologous or exogenous genes in a recombinant host microorganism. Promoters useful for this invention can come from any source (e.g., viral, bacterial, fungal, protist, animal).

Promoter elements can be selected and mobilized in a vector (e.g., pIMPC phy, see U.S. patent application Ser. No. 12/630,784, incorporated herein by reference). For example, a transcription regulatory sequence is operably linked to gene(s) of interest (e.g., in an expression construct). The promoter can be any array of DNA sequences that interact specifically with cellular transcription factors to regulate transcription of the downstream gene. The selection of a particular promoter depends on what cell type is to be used to express the protein of interest. Generally, the transcription regulatory elements from the host microorganism work quite well. In various embodiments, constitutive or inducible promoters are selected for use in a host cell. Depending on the host cell, there are potentially hundreds of constitutive and inducible promoters that are known and that can be engineered to function in the host cell. A “constitutive promoter” is a promoter that is active under most environmental and developmental conditions. An “inducible promoter” is a promoter that is active under environmental or developmental regulation.

Examples of inducible promoters/regulatory elements include, for example, a nitrate-inducible promoter (Back et al. 1991, Plant Mol. Biol.; 17(9), or a light-inducible promoter, (Feinbaum, et al. 1991, Mol. Gen. Genet.; 226:449; Lam and Chua, 1990, Science; 248:471), or a heat responsive promoter (Muller et al. 1992, Gene; 111:165-73). In some instances, promoters widely utilized in recombinant technology, for example the major left and right promoters of the bacteriophage, the E. coli lac, tac, reca, trp, AraC and gal promoters, the bacteriophage PL promoter, bacteriophage T7 and SP6 promoters, beta-actin promoter, insulin promoter, baculoviral polyhedron and p10 promoter can be used. Other useful promoters are the alpha-amylase (Ulmanen, et al. 1985, J. Bacteriol.; 162:176-182) and the sigma-28-specific promoters of B. subtilis (Gilman et al. 1984, Gene Seq. 32:11-20), the promoters of the bacteriophages of Bacillus (Gryczan, 1982. In: The Molecular Biology of the Bacilli, Academic Press, Inc., NY), Streptomyces promoters (Ward et al, 1986, Mol. Gen. Genet.; 203:468-478), and the like. Exemplary prokaryotic promoters are reviewed by Glick (1987 J. Ind. Microtiot. 1:277-282), Cenatiempo (1986 Biochemie 68:505-516), and Gottesman (1984 Ann. Rev. Genet. 18:415-442).

The promoter of the hydA gene from Clostridium acetobutylicum expression is known to be regulated by the environmental pH. Furthermore, temperature-regulated promoters are also known and can be used in this invention. Therefore, in some embodiments, depending on the desired host cell, a pH-regulated or temperature-regulated promoter can be utilized with the expression constructs of the invention.

In other instances, a constitutive promoter can be utilized. Non-limiting examples of constitutive promoters include the int promoter of the bacteriophage lambda, the bla promoter of the beta-lactamase gene sequence of pBR322, hydA or thlA in Clostridium, S. coelicolor hrdF, or whiE, the CAT promoter of the chloramphenical acetyl transferase gene sequence of pPR325, Staphylococcal constitutive promoter blaZ and the like.

For expression, other regulatory elements, such as for instance a Shine-Dalgarno (SD) sequence (e.g. AGGAGG and so on including natural and synthetic sequences operable in the host cell) and a transcriptional terminator (inverted repeat structure including any natural and synthetic sequence) that are operable in the host cell (into which the coding sequence will be introduced to provide a recombinant cell of this invention) can be used with the promoters described supra.

Repressors are protein molecules that bind specifically to particular operators. Examples of repressors are the lac repressor molecule which binds to the operator of the lac promoter-operator system, while the cro repressor binds to the operator of the lambda pR promoter. Other combinations of the repressor and operator are known in the art. See, e.g., J. D. Watson et al., Molecular Biology of the Gene, p. 373 (4^{th} ed. 1987). The structure formed by the repressor and operator blocks the productive interaction of the associated promoter with RNA polymerase, thereby preventing transcription. Other molecules, termed inducers, bind to repressors, thereby preventing the repressor from binding to its operator. Thus, the suppression of protein expression by repressor molecules can be reversed by reducing the concentration of repressor (depression) or by neutralizing the repressor with an inducer.

Analogous promoter-operator systems and inducers are known in other microorganisms. In yeast, the GAL10 and GAL1 promoters are repressed by extracellular glucose, and activated by addition of galactose, an inducer. Protein GAL80 is a repressor for the system, and GAL4 is a transcriptional activator. Binding of GAL80 to galactose prevents GAL80 from binding GAL4. Then, GAL4 can bind to an upstream activation sequence (UAS) activating transcription. See Y. Oshima, “Regulatory Circuits For Gene Expression: The Metabolisms Of Galactose And Phosphate” in The Molecular Biology Of The Yeast Saccharomyces, Metabolism And Gene Expression, J. N. Strathern et al. eds. (1982).

Transcription under the control of the PHOS promoter is repressed by extracellular inorganic phosphate, and induced to a high level when phosphate is depleted. R. A. Kramer and N. Andersen, “Isolation of Yeast Genes with mRNA Levels Controlled By Phosphate Concentration”, Proc. Nat. Acad. Sci. U.S.A., 77:6451-6545 (1980). A number of regulatory genes for PHOS expression have been identified, including some involved in phosphate regulation.

Mateα2 is a temperature-regulated promoter system in yeast. A repressor protein, operator and promoter sites have been identified in this system. A. Z. Sledziewski et al., “Construction Of Temperature-Regulated Yeast Promoters Using The Matα2 Repression System”, Bio/Technology, 6:411-16 (1988).

Another example of a repressor system in yeast is the CUP1 promoter, which can be induced by Cu⁺² ions. The CUP1 promoter is regulated by a metallothionine protein. J. A. Gorman et al., “Regulation of the Yeast Metallothionine Gene”, Gene, 48:13-22 (1986).

Similarly, to obtain desired expression of one or more cellulases, xylanases or other hydrolases, a higher copy number plasmid can be utilized in a product of process of the invention. Constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target microorganism. This DNA can be ligated to form circles without replicons and used for transformation. Thus, the gene of interest can be introduced in a heterologous host such as E. coli, and short, random fragments can be isolated and operably linked to target genes (e.g., genes encoding cellulase enzymes) to promote homologous recombination.

In one embodiment a Clostridium strain, such as C. phytofermentan, is transformed by electroporation. In one embodiment a Clostridium strain is transformed with a nucelice acid sequence that provides erythromycin resistance. In one embodiment a cell suspension is spotted onto QM1 agar plates and incubated anaerobically at 20-40 C for 4-48 hours. In another embodiment a cell suspension is spotted onto QM1 agar plates and incubated anaerobically at about 30° C. for about 24 hours. In one embodiment transformants are spotted on an agar plate, such as a QM plate, which contains erythromycin. In another embodiment spotted cells can removed from the agar plate and placed on solid or in liquid media containing antibiotics that allow the survival of transformed cells expressing erythromycin resistance. In one embodiment the solid or liquid media comprises a combination of antibiotics such as trimethoprim, cycloserine, and/or erythromycin.

In other embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized microorganisms on substrates that comprise carbon sources that will be utilized during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

In one embodiment, a product for production of a chemical product comprises: a carbonaceous biomass, an organism that is capable of direct hydrolysis and fermentation of the biomass to the product, wherein the product is, for example, the compounds described in paragraphs 69 through 71 supra, or the like.

In another embodiment, a product for production of a biofuel comprises: a carbonaceous biomass, an organism that is capable of direct hydrolysis and fermentation of the biomass, wherein said organism is modified to provide enhanced production of a chemical product such as, but not limited to, for example, the compounds described in paragraphs 69 through 71 supra, or the like.

In yet a further embodiment, a product for production of fermentive end-products comprises: (a) a fermentation vessel comprising a carbonaceous biomass; (b) and a modified organism that is capable of direct hydrolysis and fermentation of the biomass; wherein the fermentation vessel is adapted to provide suitable conditions for fermentation of one or more carbohydrates into fermentive end-products.

An organism utilized in products or processes of the invention, can be one that is capable of direct fermentation of C5 and C6 carbohydrates. In one embodiment, such a capability is achieved through modifying the organism to express one or more genes encoding proteins associated with C5 and C6 carbohydrate metabolism.

Organisms useful in compositions and methods of the invention include but are not limited to bacteria, yeast or fungi that can hydrolyze and ferment feedstock or biomass. In some embodiments, two or more different organisms can be utilized during saccharification and/or fermentation processes to produce a end-product. Organisms utilized will be recombinant.

In one embodiment, an organism utilized in compositions or methods of the invention is a strain of Clostridia. In a further embodiment, the organism is Clostridium phytofermentans.

Organisms of the invention can be modified to comprise one or more heterologous or exogenous polynucleotides that enhance enzyme function. In one embodiment, enzymatic function is increased for one or more cellulase enzymes.

An organism that utilized in products and processes of the invention can be capable of uptake of one or more complex carbohydrates from biomass (e.g., biomass comprises a higher concentration of oligomeric carbohydrates relative to monomeric carbohydrates).

In some embodiments, one or more enzymes are utilized in products and processes of the invention, which are added externally (e.g., enzymes provided in purified form, cell extracts, culture medium or commercially available source).

Enzyme activity can also be enhanced by modifying conditions in a reaction vessel, including but not limited to time, pH of a culture medium, temperature, concentration of nutrients and/or catalyst, or a combination thereof. A reaction vessel can also be configured to separate one or more desired end-products.

Products or processes of the invention provide hydrolysis of biomass resulting in a greater concentration of cellobiose relative to monomeric carbohydrates. Such monomeric carbohydrates can comprise xylose and arabinose.

In some embodiments of the present invention, batch fermentation with an organism of the invention and of a mixture of hexose and pentose saccharides using processes of the present invention provides uptake rates of about 0.1, 0.2, 0.4, 0.5, 0.6 0.7, 0.8, 1, 2, 3, 4, 5, or about 6 g/L/h or more of hexose (e.g. glucose, cellulose, cellobiose etc.), and about 0.1, 0.2, 0.4, 0.5, 0.6 0.7, 0.8, 1, 2, 3, 4, 5, or about 6 g/L/h or more of pentose (xylose, xylan, hemicellulose etc.). In one embodiment, C. phytofermentans is capable of direct fermentation of C5 and C6 sugars.

Biomass

In some embodiments, an organism is contacted with pretreated or non-pretreated biomass containing cellulosic, hemicellulosic, and/or lignocellulosic material. Additional nutrients can be present or added to the biomass material to be processed by the microorganism including nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, and/or mineral supplements. In some embodiments, one or more additional lower molecular weight carbon sources can be added or be present such as glucose, sucrose, maltose, corn syrup, lactic acid, etc. Such lower molecular weight carbon sources can serve multiple functions including providing an initial carbon source at the start of the fermentation period, help build cell count, control the carbon/nitrogen ratio, remove excess nitrogen, or some other function.

Some embodiments employ aerobic/anaerobic cycling for the bioconversion of cellulosic/lignocellulosic material to fuels and chemicals. In some embodiments, the anaerobic microorganism can ferment biomass directly without the need of a pretreatment. In certain embodiments, feedstocks are contacted with biocatalysts capable of breaking down plant-derived polymeric material into lower molecular weight products that can subsequently be transformed by biocatalysts to fuels and/or other desirable chemicals.

In some embodiments, the invention provides for a process for simultaneous saccharification and fermentation of cellulosic solids from biomass into a fermentive end-product. The process comprises treating the biomass in a closed container with an organism under conditions where the organism produces saccharolytic enzymes sufficient to substantially convert the biomass into monosaccharides and disaccharides.

Alternatively, the process comprises treating the biomass in a container with the organism and adding one or more enzymes before, concurrent or after contacting the biomass with the organism, wherein the enzymes added aid in the breakdown or detoxification of carbohydrates or lignocellulosic material.

Examples of second cultures include but are not limited to Saccharomyces cerevisiae, Clostridia species such as C. thermocellum, C. acetobutylicum, and C. cellovorans, and Zymomonas mobilis.

In some embodiments, the invention provides a process of producing a chemical compound from a lignin-containing biomass. The process comprises: 1) contacting the lignin-containing biomass with an aqueous alkaline solution at a concentration sufficient to hydrolyze at least a portion of the lignin-containing biomass; 2) neutralizing the treated biomass to a pH between 5 to 9 (e.g. 5.5, 6, 6.5, 7, 7.5, 8, 8.5, or 9); 3) treating the biomass in a closed container with a Clostridium phytofermentans bacterium under conditions wherein the Clostridium phytofermentans, optionally with the addition of one or more enzymes to the container, substantially converts the treated biomass into monosaccharides and disaccharides, and then to one or more fermentation end-products; and 4) optionally, introducing a culture of a second microorganism wherein the second organism is capable of substantially converting the monosaccharides and disaccharides into a fermentive end-product.

Modification to Introduce or Enhance Enzyme Activity

In various embodiments of the invention, one or more modification of conditions for hydrolysis and/or fermentation are implemented to enhance end-product production. Examples of such modifications include genetic modification to enhance enzyme activity in an organism that already comprises genes for encoding one or more target enzymes, introducing one or more heterogeneous nucleic acid molecules into a host organism to express and enhance activity of an enzyme not otherwise expressed in the host, modifying physical and chemical conditions to enhance enzyme function (e.g., modifying and/or maintaining a certain temperature, pH, nutrient concentration, temperature), or a combination of one or more such modifications. Other embodiments include overexpression of an endogenous nucleic acid molecule into the host organism to express and enhance activity of an enzyme already expressed in the host or to express activity of an enzyme in the host when the enzyme would not normally be expressed in the naturally-occurring host organism.

Genetic Modification

In one embodiment, methods and compositions of the invention comprise genetically modifying an organism to enhance enzyme activity of one or more enzymes, including but not limited to a metabolic intermediate. Examples of such modifications include modifying endogenous nucleic acid regulatory elements to increase expression of one or more enzymes (e.g., operably linking a gene encoding a target enzyme to a strong promoter), introducing into an organism additional copies of nucleic acid molecules to provide enhanced activity of an enzyme, operably linking genes encoding one or more enzymes to an inducible promoter or a combination thereof.

Various organisms of the invention can be modified to enhance activity of one or more enzymes to produce novel chemicals. Furthermore, an organism other than C. phytofermentans can be modified to express and/or overexpress any of these enzymes. For example, other bacteria or yeast can be modified through conventional recombinant technology to express enzymes.

Other modifications can be made to enhance end-product production of a recombinant organism of the subject invention. For example, the host can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of chemical production can be achieved.

A variety of promoters can be utilized to drive expression of the heterologous genes in a recombinant host organism, e.g., constitutive promoters, inducible promoters, for example the promoters described in paragraphs 96 through 98. The skilled artisan, having the benefit of the instant disclosure, will be able to readily choose and utilize any one of the various promoters available for this purpose.

Similarly, skilled artisans, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and operably linked to target genes (e.g., genes encoding cellulase enzymes) to promote homologous recombination.

In other embodiments, an organism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized organisms on substrates that comprise carbon sources that will be utilized during the generation of end-products by fermentation. Screening can also include measuring the production of end-products during growth of the organism, or measuring the digestion or assimilation of the carbon source(s). The isolates so obtained can further be transformed with recombinant polynucleotides or used in combination with any of the methods and compositions provided herein to further enhance biofuel production.

### Biofuel Plant and Process of Producing Biofuel:

Large Scale Ethanol Production from Biomass

Generally, there are two basic approaches to producing fuel grade ethanol from biomass on a large scale utilizing of microbial cells, especially C. phytofermentans cells. In the first method, one first hydrolyzes a biomass material that includes high molecular weight carbohydrates to lower molecular weight carbohydrates, and then ferments the lower molecular weight carbohydrates utilizing of microbial cells to produce ethanol. In the second method, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the microbial cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the below described embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, the invention features a fuel plant that includes a hydrolysis unit configured to hydrolyze a biomass material that includes a high molecular weight carbohydrate, a fermentor configured to house a medium with Clostridium phytofermentans cells or another C5/C6 hydrolyzing organism dispersed therein, and one or more product recovery system(s) to isolate a product or products and associated by-products and co-products.

In another aspect, the invention features methods of making a product or products that include combining Clostridium phytofermentans cells or another C5/C6 hydrolyzing organism and a biomass feed in a medium, and fermenting the biomass material under conditions and for a time sufficient to produce a biofuel, chemical product or fermentive end-products, e.g. ethanol, propanol, hydrogen, lignin, terpenoids, and the like as described in paragraph 0063.

In another aspect, the invention features products made by any of the processes described herein.

### Large Scale Chemical Production From Biomass

Generally, there are two basic approaches to producing chemical products from biomass on a large scale utilizing microorganisms such as Clostridium phytofermentans or other C5/C6 hydrolyzing organisms. In all methods, depending on the type of biomass and its physical manifestation, one of the processes can comprise a milling of the carbonaceous material, via wet or dry milling, to reduce the material in size and increase the surface to volume ratio (physical modification).

In a first method, one first hydrolyzes a biomass material that includes high molecular weight carbohydrates to delignify it or to separate the carbohydrate compounds from noncarbohydrate compounds. Using any combination of heat, chemical, and/or enzymatic treatment, the hydrolyzed material can be separated to form liquid and dewatered streams, which may or may not be separately treated and kept separate or recombined, and then ferments the lower molecular weight carbohydrates utilizing Clostridium phytofermentans cells or another C5/C6 hydrolyzing organism to produce one or more chemical products. In the second method, one ferments the biomass material itself without heat, chemical, and/or enzymatic pretreatment. In the first method, hydrolysis can be accomplished using acids (e.g. sulfuric or hydrochloric acids), bases (e.g. sodium hydroxide), hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic materials more exposed to any C5/C6 hydrolyzing organism, such as C. phytofermentans, which can increase fermentation rate and yield. Hydrolysis and/or steam treatment of the biomass can, e.g., produce by-products or co-products which can be separated or treated to improve fermentation rate and yield, or used to produce power to run the process, or used as products with or without further processing. Removal of lignin can, e.g., provide a combustible fuel for driving a boiler. Gaseous, e.g., hydrogen and CO₂, liquid, e.g. ethanol and organic acids, and solid, e.g. lignin, products of the fermentation can be captured and purified if desired, or disposed of, e.g., by burning. For example, the hydrogen gas can be flared, or used as an energy source in the process, e.g., to drive a steam boiler, e.g., by burning. Products exiting the fermentor can be further processed, e.g. ethanol may be transferred to distillation and rectification, producing a concentrated ethanol mixture or solids may be separated for use to provide energy or as chemical products. It is understood that other methods of producing fermentive end products or biofuels can incorporate any and all of the processes described as well as additional or substitute processes that may be developed to economically or mechanically streamline these methods, all of which are meant to be incorporated in their entirety within the scope of this invention.

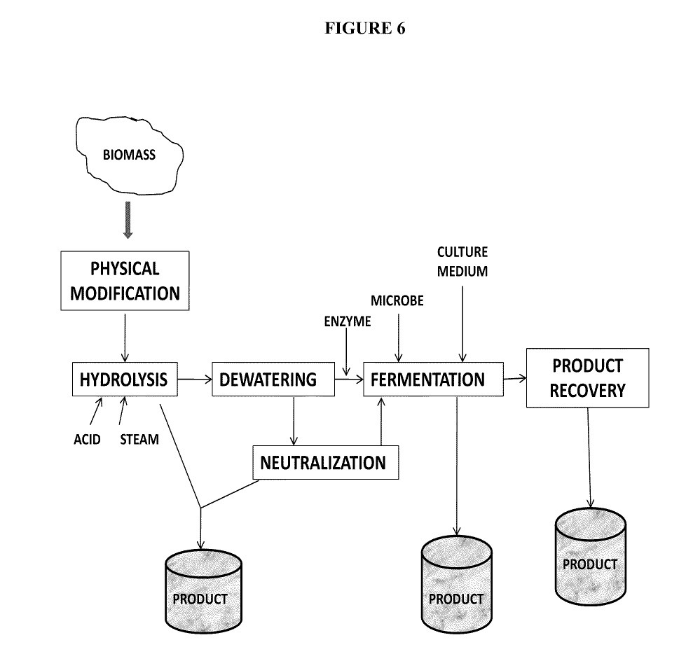

FIG. 6 is an example of a method for producing chemical products from biomass by first treating biomass with an acid at elevated temperature and pressure in a hydrolysis unit. The biomass can first be heated by addition of hot water or steam. The biomass can be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars. Steam explosion may also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass can be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products can be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

The fermentor is fed with hydrolyzed biomass, any liquid fraction from biomass pretreatment, an active seed culture of Clostridium phytofermentans cells, if desired a co-fermenting microbe, e.g., yeast or E. coli, and, if required, nutrients to promote growth of Clostridium phytofermentans or other microbes. Alternatively, the pretreated biomass or liquid fraction can be split into multiple fermentors, each containing a different strain of Clostridium phytofermentans and/or other microbes, and each operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas can be collected and used as a power source or purified as a co-product.

After fermentation, the contents of the fermentor are transferred to product recovery. Products are extracted, e.g., ethanol is recovered through distilled and rectification.

Chemical Production From Biomass Without Pretreatment

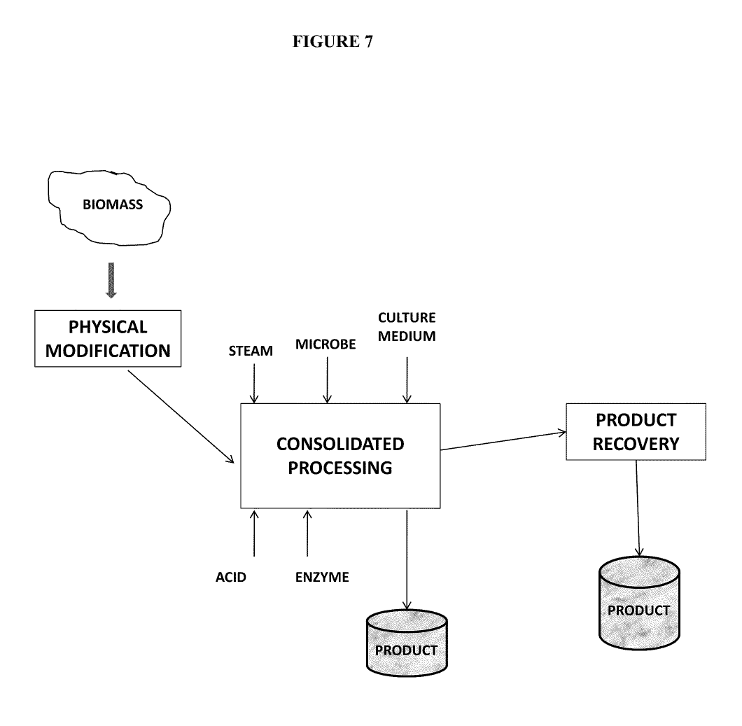

FIG. 7 depicts a method for producing chemicals from biomass by charging biomass to a fermentation vessel. The biomass can be allowed to soak for a period of time, with or without addition of heat, water, enzymes, or acid/alkali. The pressure in the processing vessel can be maintained at or above atmospheric pressure. Acid or alkali can be added at the end of the pretreatment period for neutralization. At the end of the pretreatment period, or at the same time as pretreatment begins, an active seed culture of Clostridium phytofermentans cells or another C5/C6 hydrolyzing organism and, if desired, a co-fermenting microbe, e.g., yeast or E. coli, and, if required, nutrients to promote growth of Clostridium phytofermentans or other microbes are added. Fermentation is allowed to proceed as described above. After fermentation, the contents of the fermentor are transferred to product recovery as described above.

Any combination of the chemical production methods and/or features can be utilized to make a hybrid production method. In any of the methods described herein, products can be removed, added, or combined at any step. Clostridium phytofermentans can be used alone, or synergistically in combination with one or more other microbes (e.g. yeasts, fungi, or other bacteria). Different methods can be used within a single plant to produce different products.

In another aspect, the invention features a fuel plant that includes a hydrolysis unit configured to hydrolyze a biomass material that includes a high molecular weight carbohydrate, and a fermentor configured to house a medium and contains Clostridium phytofermentans cells dispersed therein.

In another aspect, the invention features methods of making a fuel or fuels that include combining Clostridium phytofermentans cells and a lignocellulosic material (and/or other biomass material) in a medium, and fermenting the lignocellulosic material under conditions and for a time sufficient to produce a fuel or fuels, e.g., ethanol, propanol and/or hydrogen or another chemical compound.

In some embodiments, the present invention provides a process for producing ethanol and hydrogen from biomass using acid hydrolysis pretreatment. In some embodiments, the present invention provides a process for producing ethanol and hydrogen from biomass using enzymatic hydrolysis pretreatment. Other embodiments provide a process for producing ethanol and hydrogen from biomass using biomass that has not been enzymatically pretreated. Still other embodiments disclose a process for producing ethanol and hydrogen from biomass using biomass that has not been chemically or enzymatically pretreated, but is optionally steam treated.

In another aspect, the invention features products made by any of the processes described herein.

Microorganism with Reduced Lactate Dehydrogenase Activity

Modification of a mesophilic microorganism to disrupt the expression of one or more lactate dehydrogenase gene. The organism can be a naturally-occurring mesophilic organism or a mutated or recombinant organism. The term “wild-type” refers to any of these organisms with lactate dehydrogenase activity that is normal for that organism. A non “wild-type” LDH knockout is the wild-type organism that has been modified to reduce or eliminate lactate dehydrogenase activity compared to the wild-type activity level of that enzyme.

Inactivating the lactate dehydrogenase gene helps prevent the breakdown of pyruvate into lactate, and therefore promotes, under appropriate conditions, the breakdown of pyruvate into ethanol using pyruvate decarboxylase and alcohol dehydrogenase. In one embodiment, one or more naturally-occurring lactate dehydrogenase gene is disrupted by a deletion within or of the gene. In another embodiment, lactate dehydrogenase is reduced or eliminated by a chemically-induced or naturally-occurring mutation.

The wild-type microorganism may be any microorganism, mesophilic or thermophilic. In one embodiment, the microorganism is a Clostridium species. In another embodiment, it is C. phytofermentans. The microorganism can also be cellulolytic or xylanolytic or both. It can be gram negative or gram positive. In some instances, it will be anaerobic.

The microorganisms selected for modification are said to be “wild-type” and are useful in the fermentation of carbonaceous biomass. In one example, the microorganisms can be mutants or strains of Clostridium sp. and are mesophilic, anaerobic, C5/C6 saccharifying microorganisms. The microorganisms can be isolated from environmental samples expected to contain mesophiles. Isolated wild-type microorganisms will have the ability to produce ethanol but, unmodified, lactate is likely to be a fermentation product. The isolates are also selected for their ability to grow on hexose and/or pentose sugars, and oligomers thereof, at mesophilic (10° C. to 40° C.) temperatures.

In most instances, the microorganism of the invention has characteristics that permit it to be used in a fermentation process. In addition, the microorganism should be stable to at least 6% ethanol and should have the ability to utilize C₃, C₅ and C₆ sugars (or their oligomers) as a substrate, including cellobiose and starch. In one embodiment, the microorganism can saccharify C5 and C6 polysaccharides as well as ferment oligomers of these polysaccharides and monosaccharides. Furthermore, the microorganism can produce ethanol in a yield of at least 50 g/l over a 5-8 day fermentation.

The microorganism can be a spore-former or may not sporulate. The success of the fermentation process does not depend necessarily on the ability of the microorganism to sporulate, although in certain circumstances it may be preferable to have a sporulator, when it is desirable to use the microorganism as an animal feed-stock at the end of the fermentation process. This is due to the ability of sporulators to provide a good immune stimulation when used as an animal feed-stock. Spore-forming microorganisms also have the ability to settle out during fermentation, and therefore can be isolated without the need for centrifugation. Accordingly, the microorganisms can be used in an animal feed-stock without the need for complicated or expensive separation procedures.

The nucleic acid sequence for a lactate dehydrogenase can be used to target the lactate dehydrogenase gene to inactivate the gene through different mechanisms. The lactate dehydrogenase gene can be inactivated either by the insertion of a transposon, or by the deletion of the gene sequence or a portion of the gene sequence. Deletion is better, as this avoids the problem of reactivation of the gene sequence which is often experienced when transposon inactivation is used. In one embodiment, the lactate dehydrogenase gene is inactivated by the integration of a plasmid that achieves natural homologous recombination or integration between the plasmid and the microorganism's chromosome. Chromosomal integrants can be selected for on the basis of their resistance to an antibacterial agent (for example, kanamycin). The integration into the lactate dehydrogenase gene may occur by a single cross-over recombination event or by a double (or more) cross-over recombination event.

In another embodiment, the micro-organism comprises a heterologous alcohol dehydrogenase gene and a heterologous pyruvate decarboxylase gene. The expression of these heterologous genes results in the production of enzymes which redirect the metabolism so that ethanol is the primary fermentation product. These genes may be obtained from micro-organisms that typically undergo anaerobic fermentation, including Zymomonas species, including Zymomonas mobilis.

For all DNA constructs of this invention, an effective form is an expression vector. In one embodiment, the DNA construct is a plasmid or vector. In another embodiment, the plasmid comprises the nucleic acid sequence of SEQ ID NO: 1. In another embodiment, the plasmid comprises a nucleic acid with 70-99.9% similarity to the sequence of SEQ ID NO: 1. In another embodiment, the plasmid comprises a nucleic acid with 70% similarity to the sequence of SEQ ID NO: 1. In another embodiment, the plasmid comprises a nucleic acid with 75% similarity to the sequence of SEQ ID NO:1. In another embodiment, the plasmid comprises a nucleic acid with 80% similarity to the sequence of SEQ ID NO:1. In another embodiment, the plasmid comprises a nucleic acid with 85% similarity to the sequence of SEQ ID NO:1. In another embodiment, the plasmid comprises a nucleic acid with 90% similarity to the sequence of SEQ ID NO:1. In another embodiment, the plasmid comprises a nucleic acid with 95% similarity to the sequence of SEQ ID NO:1. In another embodiment, the plasmid comprises a nucleic acid with 99% similarity to the sequence of SEQ ID NO:1. In a further embodiment, the DNA construct can only replicate in the host microorganism through recombination with the genome of the host microorganism.

The DNA constructs of the invention can also incorporate a suitable reporter gene as an indicator of successful transformation. In one embodiment, the reporter gene is an antibiotic resistance gene, such as a kanamycin, ampicillin or chloramphenicol resistance gene. The DNA constructs can also incorporate multiple reporter genes, as appropriate.

Methods for the preparation and incorporation of these genes into microorganisms are known, for example in Ingram et al, Biotech & BioEng, 1998; 58 (2+3): 204-214 and U.S. Pat. No. 5,916,787, the content of each being incorporated herein by reference. The genes may be introduced in a plasmid or integrated into the chromosome, as will be appreciated by a person skilled in the art.

The microorganisms of the invention may be cultured under conventional culture conditions, depending on the mesophilic microorganism chosen. The choice of substrates, temperature, pH and other growth conditions can be selected based on known culture requirements, for example see WO01/49865 and WO01/85966, the content of each being incorporated herein by reference.

In one embodiment a recombinant organism wherein the organism lacks expression of LDH or demonstrates reduced synthesis of lactate is useful for the biofuel processes disclosed herein. In one embodiment, the recombinant microorganism used for the biofuel processes is C. phytofermentans demonstrating little or no expression of LDH. In another embodiment, a recombinant microorganism used for the biofuel processes is C. phytofermentans showing lactic acid synthesis of 100-90%, 90-80%, 80-70%, 70-60%, 60-50%, 50-40%, 40-30%, 30-20%, 20%-10%, or lower, compared to the wild-type organism. In another embodiment, a recombinant microorganism used for the biofuel processes is C. phytofermentans lacking LDH activity.

In one embodiment a microorganism engineered to knockout or reduce naturally-occurring lactate dehydrogenase is useful for producing ethanol and other chemical products, fermentive end products and/or biofuels at a higher yield than that of natural, wild-type microorganism. In one embodiment, a genetically modified microorganism such as a Clostridium species expressing reduced yields of lactic acid ose produces ethanol at a rate measurably faster than a corresponding wild-type microorganism, such as a Clostridium species that does not incorporate LDH knockout DNA construct. In one embodiment, a genetically modified Clostridium expressing an LDH knockout DNA construct ferments cellulose to a chemical product or fermentive end product more efficiently. In one embodiment, a Clostridium is engineered to express an LDH knockout DNA construct, such as SEQ ID NO:2, SEQ ID NO:3 SEQ ID NO:4, SEQ ID NO:5 or a corresponding DNA construct. In one embodiment, the chemical product is ethanol. In one embodiment, the genetically modified microorganism is a Clostridium. In another embodiment, the genetically modified microorganism is C. phytofermentans.

In one embodiment, a genetically modified microorganism comprises one or more heterologous genes in addition to an LDH knockout DNA construct. In one embodiment, the heterologous gene is a cellulase or xylanase. In another embodiment, the genetically modified microorganism that is further transformed is a Clostridium strain. In one embodiment the Clostridium strain is C. phytofermentans.

In another embodiment, the heterologous gene is an acetic acid or formic acid knockout DNA construct. In a further embodiment, the acetic acid knockout DNA construct comprises all or part of: a phosphotransacetylase (PTA) gene, such as Cphy_{—}1326, an acetyl kinase gene, such as Cphy_{—}1327, and/or a pyruvate formate lyase gene such as Cphy_{—}1174. In another embodiment, the genetically modified microorganism that is further transformed is a Clostridium strain. In one embodiment the Clostridium strain is C. phytofermentans.

In another embodiment, a biofuel plant or process disclosed herein is useful for producing biofuel with a microorganism engineered to knockout or reduce naturally-occurring lactate dehydrogenase (LDH knockout). An LDH knockout is useful for increasing yields of ethanol or other biofuels, or other chemical products from the hydrolysis of biomass in comparison to other mesophilic fermenting microorganisms. In one embodiment, a mesophilic LDH knockout can be used for reducing the amount of lactic acid in the yield of ethanol or other biofuels or fermentive end products.

In one embodiment, an LDH knockout construct can be expressed in a microorganism that does not express pyruvate carboxylase. In another embodiment, an LDH knockout construct can be expressed in a microorganism that does not produce ethanol as a primary product of its metabolic process. A microorganism that does not produce ethanol as a primary product can be a naturally occurring, or a genetically modified microorganism. For example, in a microorganism producing ethanol, lactic acid and acetic acid, the microorganism can be engineered to produce undetectable amount of lactic acid and acetic acid. The microorganism can further be engineered to express an acetic acid knockout and/or a formic acid knockout.

Methods and compositions described herein are useful for obtaining increased fermentive yields. In one embodiment, increased fermentive yield activity is obtained by transforming a microorganism with an LDH knockout construct. In another embodiment, the microorganism is selected from the group of Clostridia. In another embodiment, the microorganism is a strain selected from C. phytofermentans.

The wild-type strain of C. phytofermentans and eight lactate dehydrogenase derivative strains (LDH knockout strains) were deposited in the AGRICULTURAL RESEARCH SERVICE CULTURE COLLECTION (NRRL) (International Depositary Authority), National Center for Agricultural Utilization Research, Agricultural Research Service, U.S. Department of Agriculture, 1815 North University Street, Peoria, Ill. 61604 U.S.A. on Mar. 9, 2010 in accordance with and under the provisions of the Budapest Treaty for the International Recognition of the Deposit of Microorganisms for the Purpose of Patent Procedure. The strains were tested by the NRRL and determined to be viable. The NRRL has assigned the following NRRL deposit accession numbers to strains: C. phytofermentans Q8 (NRRL B-B-50351), C. phytofermentans 1117-1 (NRRL B-50352), C. phytofermentans 1117-2 (NRRL B-50353), C. phytofermentans 1117-3 (NRRL B-503554), C. phytofermentans 1117-4 (NRRL B-50355), C. phytofermentans 1232-1 (NRRL B-50356), C. phytofermentans 1232-4 (NRRL B-50357), C. phytofermentans 1232-5 (NRRL B-50358), and C. phytofermentans 1232-6 (NRRL B-50359).

### EXAMPLES

### Example 1

### Knockout of LDH Gene

Metabolic Engineering of Clostridium Phytofermentans:

Carbon Flux Shift from Lactic Acid to Ethanol One method of redirecting the carbon flux in the cell to increase ethanol production required construction of a non-replicative plasmid system and its incorporation into C. phytofermentans. A new genetic transfer system (GTS) was created to introduce exogenous DNA into species and strains of Clostridium. Using this system, lactate dehydrogenase (LDH) genes were targeted and subsequently disrupted by a single crossover event in the Clostridium genome. C. phytofermentans has two LDH genes: Cphy_{—}1232 and Cphy_{—}1117 (for gene annotations, see http://www.genome.jp/). Both genes were targeted and disrupted individually.

Fragments of LDH genes were cloned as follows:

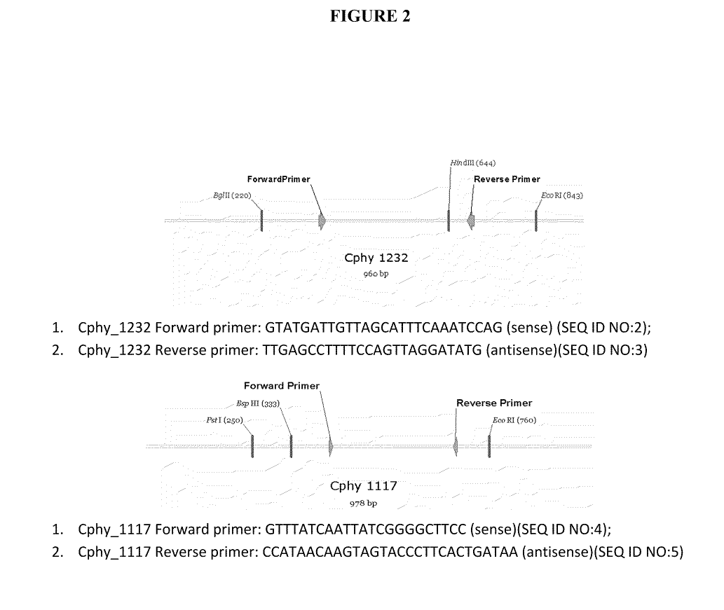

In the genus Clostridium, efficient single crossover events occur when the fragment of the gene being targeted is in the interval of 300-650 bp. Primers were designed for the amplification of both LDH's and synthesized by MWG biotech (Eurofins MWG Operon, 2211 Seminole Drive, Huntsville, Ala. 35805). The primers and their position are shown in FIG. 2.

The fragments were amplified by the polymerase chain reaction (PCR) using Phusion Polymerase (Finnzymes, Inc., 800 West Cummings Park, Suite 5550, Woburn, Mass. 01801) leaving a blunt end on either side of the PCR product. The blunt-ended products were cloned into the pCR®-Blunt II-TOPO® (Invitrogen, Inc., 5791 Van Allen Way, Carlsbad, Calif. 92008 USA).

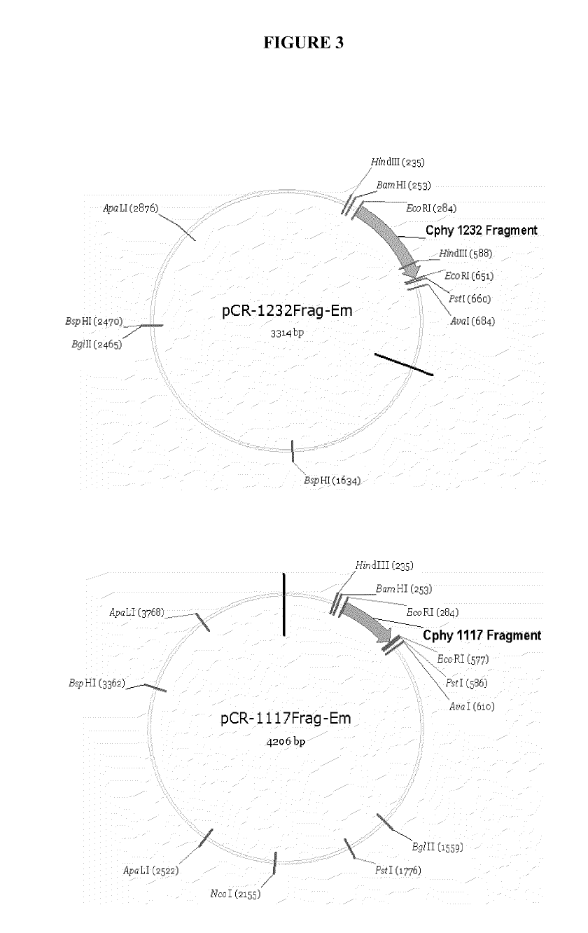

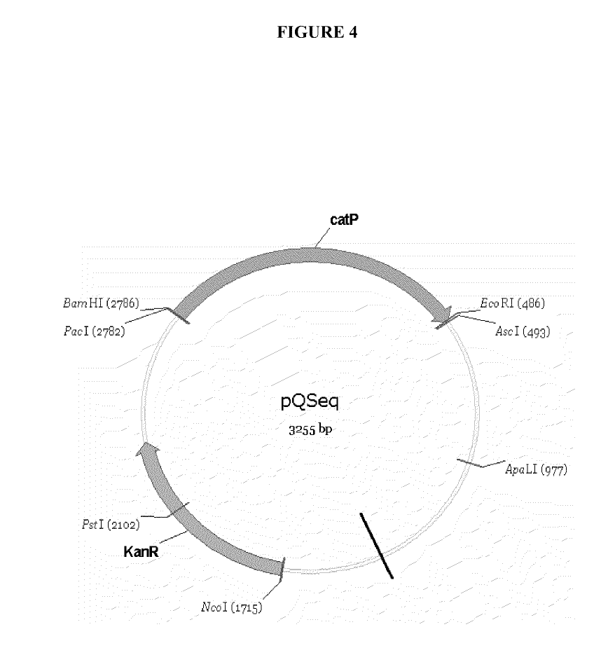

The ligation mixture comprising the two plasmid constructs (FIG. 3) were transformed into Escherichia coli—DH5α (Invitrogen, Inc) and transformants were selected on Kanamycin (50 μg/mL) X-Gal plates. A blue/white screen was utilized; white colonies indicated an interruption in the galactosidease gene, thus demonstrating a recombinant clone. White colonies were selected, cultured and plasmids were isolated using the Qiagen Plasmid Miniprep Kit (Qiagen, Inc., 27220 Turnberry Land, Valencia Calif. 91355). The LDH fragments were inserted into an engineered plasmid: “pMA-0923071.” The pMA-0923071 plasmid lacks a gram positive origin of replication, however the it does contain Chloramphenicol acetyltransferase (catP) and kanamycin acetyltransferase sites, conferring chloramphenicol and kanamycin resistance, respectively. The fully sequenced version of the plasmid is shown in FIG. 4 (pQSeq) and below.

pQSeq plasmid sequence (SEQ ID NO: 1):
accaagctatacaatatttcacaatgatactgaaacattttccagcct
ttggactgagtgtaagtctgactttaaatcatttttagcagattatga
aagtgatacgcaacggtatggaaacaatcatagaatggaaggaaagcc
aaatgctccggaaaacatttttaatgtatctatgataccgtggtcaac
cttcgatggctttaatctgaatttgcagaaaggatatgattatttgat
tcctatttttactatggggaaatattataaagaagataacaaaattat
acttcctttggcaattcaagttcatcacgcagtatgtgacggatttca
catttgccgttttgtaaacgaattgcaggaattgataaatagttaact
tcaggtttgtctgtaactaaaaacaagtatttaagcaaaaacatcgta
gaaatacggtgttttttgttaccctaaaatctacaattttatacataa
ccacgaattcggcgcgccctgggcctcatgggccttcctttcactgcc
cgctttccagtcgggaaacctgtcgtgccagctgcattaacatggtca
tagctgtttccttgcgtattgggcgctctccgcttcctcgctcactga
ctcgctgcgctcggtcgttcgggtaaagcctggggtgcctaatgagca
aaaggccagcaaaaggccaggaaccgtaaaaaggccgcgttgctggcg
tttttccataggctccgcccccctgacgagcatcacaaaaatcgacgc
tcaagtcagaggtggcgaaacccgacaggactataaagataccaggcg
tttccccctggaagctccctcgtgcgctctcctgttccgaccctgccg
cttaccggatacctgtccgcctttctcccttcgggaagcgtggcgctt
tctcatagctcacgctgtaggtatctcagttcggtgtaggtcgttcgc
tccaagctgggctgtgtgcacgaaccccccgttcagcccgaccgctgc
gccttatccggtaactatcgtcttgagtccaacccggtaagacacgac
ttatcgccactggcagcagccactggtaacaggattagcagagcgagg
tatgtaggcggtgctacagagttcttgaagtggtggcctaactacggc
tacactagaagaacagtatttggtatctgcgctctgctgaagccagtt
accttcggaaaaagagttggtagctcttgatccggcaaacaaaccacc
gctggtagcggtggtttttttgtttgcaagcagcagattacgcgcaga
aaaaaaggatctcaagaagatcctttgatatttctacggggtctgacg
ctcagtggaacgaaaactcacgttaagggattttggtcatgagattat
caaaaaggatcttcacctagatccttttaaattaaaaatgaagtttta
aatcaatctaaagtatatatgagtaaacttggtctgacagttattaga
aaaattcatccagcagacgataaaacgcaatacgctggctatccggtg
ccgcaatgccatacagcaccagaaaacgatccgcccattcgccgccca
gttcttccgcaatatcacgggtggccagcgcaatatcctgataacgat
ccgccacgcccagacggccgcaatcaataaagccgctaaaacggccat
tttccaccataatgttcggcaggcacgcatcaccatgggtcaccacca
gatcttcgccatccggcatgctcgctttcagacgcgcaaacagctctg
ccggtgccaggccctgatgttcttcatccagatcatcctgatccacca
ggcccgcttccatacgggtacgcgcacgttcaatacgatgtttcgcct
gatgatcaaacggacaggtcgccgggtccagggtatgcagacgacgca
tggcatccgccataatgctcactttttctgccggcgccagatggctag
acagcagatcctgacccggcacttcgcccagcagcagccaatcacggc
ccgcttcggtcaccacatccagcaccgccgcacacggaacaccggtgg
tggccagccagctcagacgcgccgcttcatcctgcagctcgttcagcg
caccgctcagatcggttttcacaaacagcaccggacgaccctgcgcgc
tcagacgaaacaccgccgcatcagagcagccaatggtctgctgcgccc
aatcatagccaaacagacgttccacccacgctgccgggctacccgcat
gcaggccatcctgttcaatcatactcttcctttttcaatattattgaa
gcatttatcagggttattgtctcatgagcggatacatatttgaatgta
tttagaaaaataaacaaataggggttccgcgcacatttccccgaaaag
tgccacctaaattgtaagcgttaatattttgttaaaattcgcgttaaa
tttttgttaaatcagctcattttttaaccaataggccgaaatcggcaa
aatcccttataaatcaaaagaatagaccgagatagggttgagtggccg
ctacagggcgctcccattcgccattcaggctgcgcaactgttgggaag
ggcgtttcggtgcgggcctcttcgctattacgccagctggcgaaaggg
ggatgtgctgcaaggcgattaagttgggtaacgccagggttttcccag
tcacgacgttgtaaaacgacggccagtgagcgcgacgtaatacgactc
actatagggcgaattgaaggaaggccgtcaaggccgcatttaattaag
gatccggcagtttttctttttcggcaagtgttcaagaagttattaagt
cgggagtgcagtcgaagtgggcaagttgaaaaattcacaaaaatgtgg
tataatatctttgttcattagagcgataaacttgaatttgagagggaa
cttagatggtatttgaaaaaattgataaaaatagttggaacagaaaag
agtattttgaccactactttgcaagtgtaccttgtacatacagcatga
ccgttaaagtggatatcacacaaataaaggaaaagggaatgaaactat
atcctgcaatgctttattatattgcaatgattgtaaaccgccattcag
agtttaggacggcaatcaatcaagatggtgaattggggatatatgatg
agatgataccaagctatacaatatttcacaatgatactgaaacatttt
ccagcctttggactgagtgtaagtctgactttaaatca

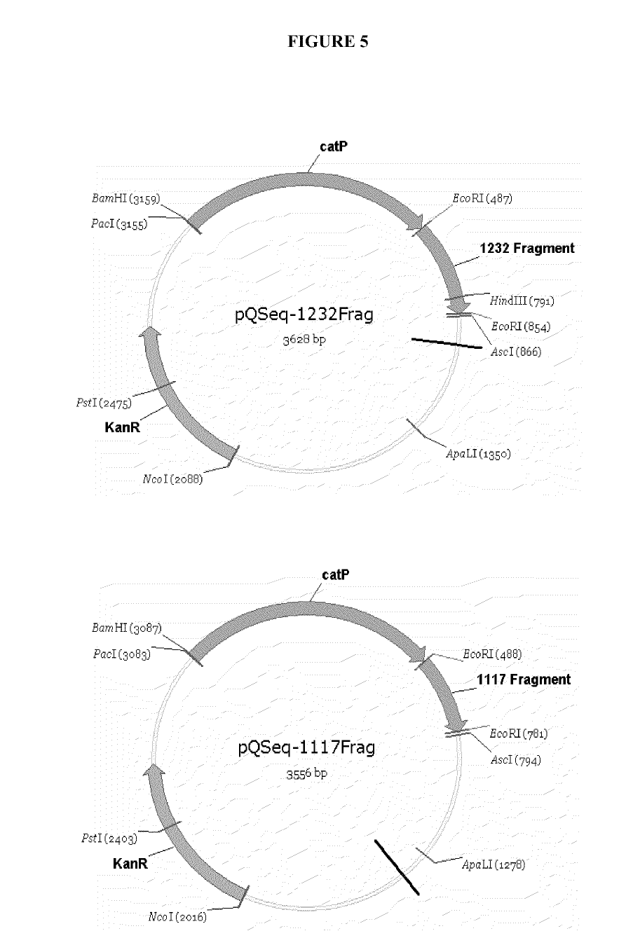

There is an EcoR1 site after the catP cassette on the plasmid. Additionally, both LDH fragments in the pCR plasmid were flanked with an EcoR1 site. Both the pCR-1232-Em and pCR1117-Em plasmids (FIG. 5) were digested with EcoR1 (New England BioLabs, Inc.), and the appropriate restriction fragment within the digested samples was size selected by agarose gel electrophoresis and extracted from the gel using Qiagen Gel Purification. The same process was used to obtain EcoR1-digested pQSeq plasmid. The EcoRI-digested pQSeq plasmid was dephosphorylated (to reduce the likelihood of relegation) using Shrimp Alkaline Phosphatase (SAP; New England BioLabs, Inc., 240 County Road, Ipswich, Mass. 01938-2723). Both LDH fragments were ligated into EcoRI-digested, dephosphorylated, pQSeq to form pQSeq-1232Frag and pQSeq-1117Frag (FIG. 5).

The ligation mixture was transformed into E. coli (DH5α). Colonies that had the ability to grow on Kanamycin (50 μg/mL) selective media were selected and subsequently cultured to isolate the plasmid. Recombinants were verified by digestion of the isolated DNA with EcoR1 and subsequent agarose gel electrophoresis to ensure the appropriate restriction fragments were present. Positive clones were stored in glycerol stocks, and a 50 ml volume was grown to isolate a minimum of 10 g of DNA for electroporation with C. phytofermentans.

The plasmids, pQSeq-1232Frag and pQSeq-1117Frag were transformed into C. phytofermentans using a standardized electroporation procedure, such as that used in U.S. application Ser. No. 12/630,784, filed on Dec. 3, 2009, which is herein incorporated by reference in its entirety. As a single crossover event is infrequent; one microgram of DNA was used per electroporation reaction to increase the likelihood of crossover selection. Table 2 shows the experimental variables from the electroporation reaction.

TABLE 2
Table 2

| Name | V | μF | Ω | Vexp | Texp |
|---|---|---|---|---|---|
| 1117-1 | 2250 | 25 | 600 | 2237 | 1.1 |
| 1117-2 | 2250 | 25 | 600 | 2231 | 4.2 |
| 1232-1 | 2250 | 25 | 600 | 2230 | 3.8 |
| 1232-2 | 2250 | 25 | 600 | 2228 | 3.7 |

The transformed cells were spread on selective agar plates (chloramphenicol at 35 g/mL). After three days, C. phytofermentans colonies were visible on the plate. All colonies were subcultured and liquid glycerol stocks were made of each.

Several colonies of C. phytofermentans further analyzed in cellobiose fermentation experiments. All shake flask experiments were conducted with selective pressure of Chloramphicol. The data from a 6% cellobiose flask experiment, conducted in triplicates, is shown below in Table 3. Ethanol (EtOH) and Lactic Acid concentrations are given in g/L.

Q8 (control) and the results of cellobiose fermentations for eight LDH-knockout strains demonstrate the effects of reduction of LDH activity. The lactic acid and ethanol production show marked shifts; ethanol titers have, on average, increased by 6-25% and lactic acid production has decreased 24-104% in the LDH knockout strains. These strains have also been subcultured and grown in corn stover as a carbon source for fermentations with and without antibiotic selectivity.

TABLE 3
Table 3

| | | Lactic | % EtOH Increase | % Lactic Acid Decrease |
|---|---|---|---|---|
| | EtOH | Acid | from Q.8 | from Q.8 |
| Q8 | 21.5 | 5.3 | 0.00 | 0.00 |
| 1117-1 | 26.6 | 2.6 | 19.07 | −104.25 |
| 1117-2 | 27.6 | 3.5 | 22.09 | −52.72 |
| 1117-3 | 28.7 | 2.6 | 25.20 | −100.51 |
| 1117-4 | 28.0 | 3.3 | 23.30 | −62.78 |
| 1232-1 | 27.2 | 4.1 | 20.93 | −28.71 |
| 1232-4 | 25.5 | 2.8 | 15.74 | −90.48 |
| 1232-5 | 23.1 | 3.3 | 6.88 | −62.00 |
| 1232-6 | 26.8 | 4.1 | 19.88 | −27.73 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. An isolated microorganism genetically modified to lack expression of a wild-type lactate dehydrogenase gene, wherein the microorganism can hydrolyze both C5 and C6 polysaccharides.

2. The microorganism of claim 1, wherein said microorganism further does not express pyruvate decarboxylase.

3. The microorganism according to claim 1, wherein said microorganism does not comprise an integration element in a lactate dehydrogenase gene.

4. The microorganism according to claim 1, wherein said microorganism is a mesophilic microorganism.

5. The microorganism according to claim 1, wherein said lactate dehydrogenase gene is Cphy_{—}1232 or Cphy_{—}1117.

6. The microorganism according to claim 1, wherein said microorganism is gram negative.

7. The microorganism according to claims 1, wherein said microorganism is gram positive.

8. The microorganism of claim 1, wherein said microorganism is a Clostridium species.

9. The microorganism of claim 8, wherein said Clostridium species is C. phytofermentans.

10. An isolated microorganism selected from the group consisting of: NRRL Accession No. NRRL B 50351, NRRL B-50352, NRRL B-50353, NRRL B 50354, NRRL B-50355, NRRL B-50356, NRRL B-50357, NRRL B-50358, and NRRL B-50359.

11. An isolated mesophilic microorganism that lacks lactate dehydrogenase activity, wherein said mesophilic microorganism ferments polysaccharides.

12. An isolated mesophilic microorganism, genetically modified to produce higher amounts of ethanol, wherein said modification comprises inactivation of lactic acid synthesis as it occurs in the wild-type mesophilic microorganism.

13. The microorganism according to claim 11 or 12, wherein said microorganism is an anaerobic microorganism that can saccharify both C5 and C6 polysaccharides.

14. The microorganism according to claim 11 or 12, wherein said microorganism is gram negative.

15. The microorganism according to claim 11 or 12, wherein said microorganism is gram positive.

16. A product for production of a chemical product comprising:
a. a fermentation vessel comprising a carbonaceous biomass; and
b. a microorganism genetically modified to inactivate a lactate dehydrogenase gene of the wild-type organism, wherein the microorganism hydrolyzes both C5 and C6 polysaccharides and produces a chemical product.

17. The product of claim 16, wherein said microorganism is capable of direct fermentation of C5 and C6 carbohydrates.

18. The product according to claim 16, wherein said microorganism is a mesophilic microorganism.

19. The product according to claims claim 16, wherein said microorganism is a Clostridium species.

20. The product according to claim 16, wherein said lactate dehydrogenase gene is Cphy_{—}1232 or Cphy_{—}1117.

21. The product according to claim 16, wherein said biomass comprises cellulosic or lignocellulosic materials.

22. The product according to claim 16, wherein said biomass comprises one or more of corn steep solids, corn steep liquor, malt syrup, xylan, cellulose, hemicellulose, fructose, glucose, mannose, rhamnose, or xylose.

23. The product according to claims claim 16, wherein said biomass comprises municipal waste, wood, plant material, plant material extract, bagasse, switch grass, algae, corn stover, poplar, a natural or synthetic polymer, or a combination thereof.

24. The product according to claim 16, wherein said biomass is pre-conditioned with acid treatment.

25. The product according to claim 16, wherein said chemical product is an alcohol, a gas, an acid, a fatty acid, an isoprenoid, or a polyisoprene.

26. The product according to claim 16, wherein said microorganism is capable of hydrolyzing xylose.

27. The product according to claim 16, wherein said chemical product is ethanol, butanol, methanol, methane, or hydrogen.

28. A process for producing a chemical product comprising:
a. contacting a carbonaceous biomass with a microorganism genetically modified to inactivate a lactate dehydrogenase gene of the wild-type organism, wherein the microorganism hydrolyzes both C5 and C6 polysaccharides;
b. allowing sufficient time for said hydrolysis and fermentation to produce the chemical product.

29. The process of claim 28, wherein said microorganism is modified to enhance production of a fermentive end-product is selected from the group consisting of Clostridium phytofermentans, Thermoanaerobacter ethanolicus, Clostridium thermocellum, Clostridium beijerinckii, Clostridium acetobutylicum, Clostridium. cellovorans, Clostridium tyrobutyricum, Clostridium thermobutyricum, Thermoanaerobacterium saccharolyticum, Thermoanaerobacter thermohydrosulfuricus, and Saccharomyces cerevisiae, Clostridium acetobutylicum, Moorella ssp., Carboxydocella ssp., Zymomonas mobilis, E. coli, and Klebsiella oxytoca.

30. The process of claim 28, wherein said microorganism is capable of direct fermentation of C5 and C6 carbohydrates.

31. The process according to claim 28, wherein said microorganism is a Clostridium.

32. The process according to claim 28, wherein said microorganism is a mesophilic microorganism.

33. The process according to claim 28, wherein said microorganism is C. phytofermentans.

34. The process according to claim 28, wherein said lactate dehydrogenase gene is Cphy_{—}1232 or Cphy_{—}1117.

35. The process according to claim 28, wherein said biomass comprises cellulosic or lignocellulosic materials.

36. The process according to claim 28, wherein said biomass comprises one or more of corn steep solids, corn steep liquor, malt syrup, xylan, cellulose, hemicellulose, fructose, glucose, mannose, rhamnose, or xylose.

37. The process according to claim 28, wherein said biomass comprises municipal waste, wood, plant material, plant material extract, bagasse, switch grass, algae, corn stover, poplar, a natural or synthetic polymer, or a combination thereof.

38. The process according to claim 28, wherein said biomass is pre-conditioned with acid treatment.

39. The process according to claim 28, wherein said process occurs at a temperature between 10° C. and 35° C.

40. A chemical product produced by the process according to claim 28.

41. The chemical product of claim 40, wherein said product is an alcohol.

42. The chemical product of claim 41, wherein said alcohol is ethanol.

43. An isolated microorganism that ferments cellulosic or lignocellulosic materials to produce ethanol in a concentration that is at least 90% of a theoretical yield, wherein the microorganism is genetically modified to inactivate a lactate dehydrogenase gene of the wild-type organism.

44. The isolated microorganism of claim 43, wherein the microorganism saccharifies C5 and C6 polysaccharides.

45. The isolated microorganism of claim 43, wherein the microorganism is gram negative.

46. The isolated microorganism of claim 43, wherein the microorganism is gram positive.

47. The isolated microorganism of claim 43, wherein said lactate dehydrogenase gene is Cphy_{—}1232 or Cphy_{—}1117.

48. A plasmid comprising SEQ ID NO:1.

49. A primer selected from the group comprising SEQ ID NO:2, SEQ ID NO:3, SEQ ID NO:4, and SEQ ID NO:5.

50. A plasmid comprising a nucleic acid sequence with about 70% similarity to SEQ ID NO:1.

51. A primer comprising a nucleic acid sequence with about 70% similarity to SEQ ID NO:2, SEQ ID NO:3, SEQ ID NO:4, or SEQ ID NO:5.
